(19) United States
(12) Patent Application Publication
(10) Pub. No.: US 2025/0099378 A1
(43) Pub. Date: Mar. 27, 2025

(54) INHALABLE FORMULATIONS

(71) Applicant: NANOPHARM LIMITED, Newport (GB)

(72) Inventors: Jagdeep SHUR, Bath (GB); Robert PRICE, Llanvair Discoed (GB)

(21) Appl. No.: 18/730,323

(22) PCT Filed: Jan. 20, 2023
(86) PCT No.: PCT/GB2023/050117

§ 371 Date: Jul. 19, 2024

(30) Foreign Application Priority Data
Jan. 21, 2022 (GB) 2200809.8

(51) Int. Cl. A61K 9/00; A61K 9/08; A61K 31/167; A61K 31/573; A61K 47/06; A61K 47/10; A61K 47/18

(52) U.S. Cl. CPC A61K 9/008; A61K 9/08; A61K 31/167; A61K 31/573; A61K 47/06; A61K 47/10; A61K 47/183

(57) ABSTRACT

An inhalable formulation comprising at least one amino acid and at least one beta-2-agonist is disclosed. The inhalable formulation may be administered by pressurized metered dose inhaler.

### FIELD OF THE INVENTION

The present invention concerns inhalable formulations. More particularly, the present invention concerns inhalable formulations comprising at least one amino acid, at least one beta2-agonist and a propellant. The inhalable formulations of the present invention have been found to be particularly suited for delivery by a pressurised metered dose inhaler (pMDI).

### BACKGROUND OF THE INVENTION

Orally inhalable formulations are widely used for the administration of medications via a pulmonary route. Such medications are generally administered for treatment or prophylaxis of pulmonary conditions, the commonest of which include, for example, asthma and chronic obstructive pulmonary disorder. Inhalable therapeutics are advantageous because they achieve rapid delivery and direct targeting of the lungs and respiratory tract by inhalation. This can minimise side effects as the drug is released directly at the site of action.

Pressurised metered dose inhalers (pMDIs) are well known devices for administering pharmaceutical compositions and formulations to the respiratory tract by inhalation. MDIs when actuated create propellant droplets containing pharmaceutical product for delivery to the respiratory tract as an aerosol. Preferred propellants for pharmaceutical use are hydrofluoroalkanes (HFAs), also known as hydrofluorocarbons. HFAs are less destructive to the ozone layer than chlorofluorocarbons (CFCs) and are a suitable alternative to CFCs.

However, HFA propellants have higher polarity compared to CFC counterparts and therefore HFA solution formulations may suffer from chemical stability problems. Fluorinated carbon compounds containing terminal protons show considerably different solubility behaviour than do those compounds which are fully halogenated or fully protonated. These terminal protons are relatively electrophilic and are capable of forming hydrogen bonds with appropriate Lewis bases, such as ketones and amines. The magnitude of these interactions are difficult to measure or predict because of overlapping effects, such as differences in intermolecular attractions between fluorine-fluorine or hydrogen-hydrogen atoms. Successful manufacture of a formulation system that provides chemical stability of the active ingredient is difficult to predict in non-polar liquids. The addition of small amounts of acids or the leaching of inactive ingredients from the valve of the MDI may also give rise to enhanced or inhibited hydrogen bonding that may impact the stability of the drug.

Beta2-agonists, such as formoterol, may be delivered to the lungs by inhalation of a solution formulation of the drug in hydrofluoroalkane propellant. This may be in combination with other drugs such as steroids (e.g. corticosteroids) and/or muscarinic antagonists. However, beta2-agonists, such as formoterol, suffer from inherent chemical stability problems. Beta2-agonists having a benzylic hydroxyl group, such as formoterol and salbutamol, suffer from inherent chemical instability due to their susceptibility to substitution by nucleophilic species in formulation via S_{N}1 or S_{N}2 reactions. Ethanol can satisfy the role of a nucleophile and cause degradation. The high polarity of HFA propellants may exacerbate the inherent stability problems associated with beta2-agonists.

Thus, a challenge associated with inhalable formulations, particularly those formulations comprising a beta2-agonist, is the chemical stability of the active ingredients over the shelf-life of the formulation.

One approach that has been implemented to stabilise inhalable formulations comprising beta2-agonists is the use of mineral acid in formulation (see, for example, EP1787639 A2, EP 1157689 A1 and WO 2007/121913 A2). It has been found that the chemical stability of beta2-agonists in HFA propellant and ethanol formulation can be improved by adjustment of the ‘apparent’ pH of the solution to between 2.5 and 5.0 by addition of small amounts of mineral acid. Preferred mineral acids are hydrochloric, nitric, and phosphoric acid. EP 2223682 B1 discloses a stabilised aerosol composition comprising an active ingredient formoterol fumarate in combination with beclometasone diproprionate, in a solution of a liquefied HFA 134a propellant and 12% w/w ethanol as a co-solvent, and hydrochloric acid in an amount such that the solution has an ‘apparent’ pH between 3.0 and 3.5. A significant problem with this approach is that in relatively aprotic solvents, such as HFA-ethanol solutions, protons are non-hydrates and their activity coefficients differ significantly from those in aqueous solution. Thus, a true pH value cannot be obtained and only an ‘apparent’ pH or acidity function can be estimated. This means that the amount of acid necessary to stabilise and formulation cannot be accurately determined. Furthermore, mineral acids may not be ideal to use in significant quantities in inhalable therapeutics due to biocompatibility issues.

An alternative approach to stabilising such formulations utilises organic acids (e.g. maleic acid and lactic acid) that have a weaker proton donating ability than mineral acids. WO2019/236559 A1 discloses pharmaceutical compositions for use with pressurized metered dose inhalers and comprising a beta2-agonist, a corticosteroid and/or a long acting muscarinic antagonist, a propellant, a co-solvent, an organic acid, and optionally water. It was found that organic acids such as maleic acid can stabilise beta2-agonists in such solution formulations. However, many organic acids, such as those described in WO2019/236559 A1, have no, or little, toxicology data with respect to exposure in the lung.

The present invention seeks to mitigate the above-mentioned problems. For example, the present invention seeks to provide stabilised inhalable formulations, particularly formulations comprising beta2-agonists.

### SUMMARY OF THE INVENTION

According to a first aspect of the invention is an inhalable formulation comprising at least one amino acid, at least one beta2-agonist comprising a benzylic hydroxyl moiety according to the structural formula:

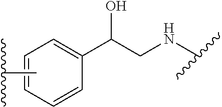

and a propellant,
wherein the inhalable formulation is advantageously a solution formulation in which the at least one beta2-agonist is dissolved in solution.

According to the first aspect of the invention, the inhalable formulation comprises at least one amino acid. It has been found that amino acids are particularly advantageous in stabilising inhalable formulations according to the present invention. In particular, amino acids have been found to stabilise the at least one beta2-agonist present in the formulation. Without wishing to be bound by theory, it is thought that the zwitterionic characteristic of the at least one amino acid stabilises the beta2-agonist. This stabilising effect may be particularly advantageous in formulations comprising a propellant in which beta2-agonists are known to be unstable, such as a hydrofluoroalkane (HFA) propellant. Zwitterionic molecules, such as amino acids, have both basic and acidic properties. Zwitterionic molecules are therefore able to control the pH of a solution by acting as a buffer in solution. The at least one amino acid may therefore control the pH and proton acceptor/donor profile in the formulation, stabilising the formulation and in particular the at least one beta2-agonist present therein.

Precise measurement of the pH of a non-aqueous formulation, such as a formulation prepared in a HFA propellant, is difficult and unreliable. Use of at least one amino acid in inhalable formulations of the present invention buffers the solution so that the pH of the formulation does not need to be accurately determined. Additionally, the formulation of the present invention does not need to be prepared to a particular pre-defined pH, for example by addition of a mineral acid such as hydrochloric acid, as the zwitterionic properties of the at least one amino acid will maintain the formulation within acceptable pH range. Advantageously, amino acids are biocompatible, and thus may be delivered into the human body, for example through the respiratory tract and lungs, without adverse reaction or side-effects. This is in contrast to some known buffer solutions which may not be suitable for therapeutic uses.

According to the first aspect of the invention, the inhalable formulation comprises at least one beta2-agonist. Beta2-agonists are effective for the treatment of respiratory tract and/or lung diseases such as chronic asthma and chronic obstructive pulmonary disease (COPD).

According to the first aspect of the invention, the beta2-agonist comprises a benzylic hydroxyl moiety according to structural Formula I;

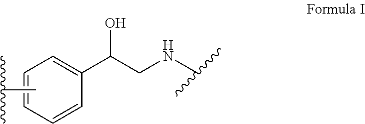

The benzylic hydroxyl moiety according to Formula I is common to many known beta2-agonists. Without wishing to be bound by theory, it is believed that the at least one amino acid in the inhalable formulation stabilises the benzylic hydroxyl group according to Formula I.

According to the first aspect of the invention, the inhalable formulation comprises a propellant. The propellant aerosolises the formulation when expelled from a pressurised metered dose inhaler.

Preferably, the at least one amino acid is selected from the list of aspartic acid, leucine, isoleucine, alanine, and/or valine, and derivatives, pharmaceutically acceptable salts and/or solvates thereof. Most preferably, the amino acid is leucine and/or aspartic acid.

Optionally, the inhalable formulation is a solution formulation. A solution formulation is a formulation in which at least one active ingredient is dissolved in solution. Preferably, when the inhalable formulation is a solution formulation, the at least one active ingredient dissolved in solution is the at least one beta2-agonist. The solvent of the solution formulation may be the propellant. The solvent may additionally comprise a co-solvent, such as ethanol, as described herein. It will be understood by the skilled person that a solution formulation is a solvent mixture in which the solute (for example, the at least one active ingredient, such as the at least one beta2-agonist) is dissolved in and uniformly distributed within the solvent (for example, the propellant). This is in contrast to dry powder or suspension formulations which comprise active ingredients in solid particle form. For the avoidance of doubt, it will be understood that solution formulations according to the present invention may additionally comprise components which are not in solution and are suspended in the solution formulation.

Preferably, an inhalable formulation according to the first aspect of the invention comprises at least one beta2-agonist selected from the list of: formoterol, indacaterol, olodaterol, salmeterol, carmoterol, and/or vilanterol, and derivatives, pharmaceutically acceptable salts and/or solvates thereof. When beta2-agonist compounds, such as formoterol, indacaterol, olodaterol, salmeterol, carmoterol, and/or vilanterol, are referred to herein by name it is to be understood that the reference encompasses the beta2-agonist in the form of a pharmaceutically acceptable salts and/or solvates of compound and also derivatives of the compound, such as prodrugs, for example esters, and pharmaceutically acceptable salts and/or solvates thereof, unless it is clear from the context that only the compound per se, e.g. the free-base, is intended.

Preferably, the at least one beta2-agonist includes formoterol or a derivative, pharmaceutically acceptable salt and/or solvate thereof. More preferably, the at least one long-acting beta2-agonist includes formoterol fumarate. Preferably, the formoterol fumarate is in the form of the dihydrate.

Preferably, the propellant comprises at least one hydrofluoroalkane (HFA). Most preferably, the HFA is selected from the list of HFA 134a, HFA 152a or HFA 227. The propellant may comprise combinations thereof.

In embodiments of the invention, the inhalable formulation may further comprise at least one steroid. Preferably, the at least one steroid is one or more inhaled corticosteroids (ICS). ICSs are particularly useful in the treatment of asthma. Formulations comprising a combination of at least one beta2-agonist and at least one ICS may be particularly effective for treating a disease of the respiratory tract and/or lungs, for example a combination of formoterol and beclometasone may be particularly effective.

Preferably, the at least one steroid is beclometasone or a derivative, pharmaceutically acceptable salt and/or solvate and combination thereof. Most preferably the at least one steroid is beclometasone dipropionate. It may be that a combination of formoterol fumarate and beclometasone dipropionate are particularly effective for treating a disease of the respiratory tract and/or lungs.

In embodiments of the invention, the inhalable formulation may further comprise at least one muscarinic antagonist. Muscarinic antagonists may alternatively be referred to as muscarinic agents. Muscarinic antagonists trigger bronchodilation and so are particularly effective for use in the treatment of a disease of the respiratory tract and/or lungs. A combination of at least one muscarinic antagonist, at least one beta2-agonist and optionally at least on steroid, preferably at least one inhaled corticosteroid, may be particularly effective for treating a disease of the respiratory tract and/or lungs. Preferably, the at least one muscarinic antagonist is one or more muscarinic antagonist selected from the list of: ipratropium bromide, oxitropium bromide, tiotropium bromide, aclidinium bromide, glycopyrronium bromide, and umeclidinium bromide, and derivatives, pharmaceutically acceptable salts and/or solvates thereof. It may be that a combination of formoterol fumarate and beclometasone dipropionate with at least one muscarinic antagonist selected from that list are particularly effective for treating a disease of the respiratory tract and/or lungs.

In embodiments of the invention, the inhalable formulation further comprises a co-solvent or mixture of co-solvents. Preferably, the co-solvent or mixture of co-solvents is a pharmaceutically acceptable alcohol or mixture of alcohols. Most preferably, the co-solvent comprises ethanol.

The inhalable formulation according to the first aspect of the invention may be particularly effective for treating and/or preventing a disease of the respiratory tract and/or lungs. Therefore, according to a second aspect, the invention provides an inhalable formulation according to the first aspect of the invention, for use in the prevention or treatment of a disease of the lungs and/or respiratory tract. The second aspect of the invention also provides the use of an inhalable formulation according to the first aspect of the invention in the manufacture of a medicament for the prevention or treatment of a disease of the lungs and/or respiratory tract. The second aspect of the invention also provides a method of the prevention or treatment of a disease of the lungs and/or respiratory tract comprising the step of administering an inhalable formulation according to the first aspect of the invention to a person in need thereof. Preferably, the inhalable formulation according to the first aspect of the invention is for use in the treatment of a disease of the lungs and/or respiratory tract.

Preferably, the inhalable formulation according to the first aspect of the invention is for use according to the second aspect of the invention wherein the disease of the lungs and/or respiratory tract is asthma or chronic obstructive pulmonary disease (COPD) such as bronchitis.

According to a third aspect, the invention provides a pressurized canister for use in a pressurised metered dose inhaler, the canister being pressurized with the formulation according to the first or second aspect of the invention. The inhalable formulation of the first or second aspect of the invention may be particularly suited for delivery by a pressurised metered dose inhaler. Therefore, according to a fourth aspect the invention provides provided a pressurised metered dose inhaler comprising the pressurized canister according to the third aspect of the invention.

It will of course be appreciated that features described in relation to one aspect of the present invention may be incorporated into other aspects of the present invention.

### DETAILED DESCRIPTION

The present invention provides an inhalable formulation. The inhalable formulation may also be referred to as an inhalable medicinal formulation. It will be understood that an inhalable formulation, or inhalable medicinal formulation, is a formulation which is suitable for administration to a human or animal patient, preferably a human patient, by inhalation. The inhalable formulation comprises one or more active ingredients that is effective in the treatment, prophylaxis or diagnosis of a disease or condition of a human or animal, especially a human, that is capable of pulmonary administration by inhalation. Inhalable formulations of the invention are formulations envisaged for use in metered dose inhalers (MDIs), including pressurised metered dose inhalers (pMDIs). Preferably, such formulations are in the form of solution formulations in which at least one active ingredient is dissolved in solution. However, it is also envisaged that the inhalable formulations of the present invention may be, without limitation, in the form of powders for use in dry powder inhalers, and solutions or suspensions for use in nebulizer devices.

“Active ingredient” is to be understood as including ingredients which are effective through any therapeutic route, this may include but is not limited to beta2-agonists, inhaled corticosteroids and muscarinic antagonists. For the avoidance of doubt, active ingredients for the purpose of this application include therapeutically effective drugs that can be administered via the pulmonary route for local treatment, prophylaxis or diagnostic methods to be practised on the lung, therapeutically effective drugs that can be administered via the pulmonary route for systemic treatment, prophylaxis or diagnostic methods to be practised on one or more other parts of the body of the patient, and active ingredients that can be administered via the pulmonary route for local treatment, prophylaxis or diagnostic methods to be practised on the lung by mechanical or physical routes, as in the case of lung surfactant. Active ingredients administered by the pulmonary route for local effect include, for example, drugs for use in the treatment of asthma, COPD, allergic rhinitis, cystic fibrosis, and tuberculosis. Systemic drugs administrable via the pulmonary route include for example insulin and small peptide therapeutics.

The inhalable formulation according to the present invention comprises at least one amino acid, at least one beta2-agonist and a propellant. Each component of the formulation is described herein. It is to be understood that any component described herein may be provided in an inhalable formulation comprising any other component described herein.

Preferably, formulations of the present invention are suitable for use in solution metered dose inhalers, for example in pressurised dose metered inhalers (pMDIs). It will be understood by the skilled person that formulations suitable for use in solution metered dose inhalers are in the form of a solution.

Preferably, formulations according to the present invention are stable at room temperature (such as between 2° and 25° C.). Preferably, formulations according to the present invention are stable at elevated temperatures, for example of 40° C. or greater, and optionally high relative humidity, for example 75% relative humidity or greater. Preferably, formulations according the present invention have an acceptable shelf-life, for example of 1 month or greater, 2 months or greater, 3 months or greater, 6 months or greater, or 1 year or greater, when stored at room temperature. Preferably, formulations of the present invention may be stable for 1 month or greater, 2 months or greater, 3 months or greater, 6 months or greater, or 1 year or greater, when stored at 40° C. and a relative humidity of 75%. The stability of a formulation is measured from the point that the formulation is manufactured. The stability of formulation may be expressed as the amount of active ingredients remaining in the formulation after a specified period of time, relative to the amount of that active ingredient in the formulation at the point of manufacture, typically expressed as a percentage of active ingredient remaining in the formulation after a specified period of time. Stability of the formulation may be affected by the conditions of storage, for example, temperature, humidity and light conditions. It may be expected that a formulation stored at elevated temperature (for example, 40° C. or greater) may degrade more quickly than a formulation stored at room temperature (for example 20° C.) or at a lower temperature (for example, 8° C. or less). High humidity and exposure to light, particularly UV light may be expected to reduce the shelf life of the formulation.

The inhalable formulation may be provided in the form of suspension in a propellant, such that at least one an active ingredient, such as formoterol, is provided in micronized form in a propellant, such as a HFA. When the formulation is provided in the form of a suspension in a propellant, the drug may be inhaled in aerosol form (a dispersion of solid particles in a gaseous medium). Such an aerosol may be produced by use of a pMDI which is used to expel the formulation. Alternatively, the formulation may be a solution formulation in which at least one active ingredient is dissolved in solution. Optionally, where more than one active ingredient is present, all active ingredients of the solution formulation are dissolved in solution. Optionally, the solution formulation comprises additional components which are suspended in the solution.

### Amino Acid

The inhalable formulation of the present invention comprises at least one amino acid. The inhalable formulation may comprise one amino acid, or may comprise a combination of two or more amino acids.

Preferably, the at least one amino acid is a naturally occurring amino acid. That is an amino acid selected from the list of alanine, arginine, asparagine, aspartic acid, cysteine, glutamic acid, glutamine, glycine, histidine, isoleucine, leucine, lysine, methionine, phenyl alanine, proline, serine, threonine, tryptophan, tyrosine, and valine. Advantageously, such amino acids are biocompatible. Amino acids therefore provide an advantage over other inorganic acids (such as hydrochloric acid or nitric acid) or organic acids (such as maleic acid) which are either not biocompatible or their bio-safety is unknown, particularly in relation to the human respiratory system. A mixture of any amino acids listed herein may be provided in the inhalable formulation according to the present invention.

Preferably, the at least one amino acid is selected from the list of aspartic acid, leucine, isoleucine, alanine, and/or valine, and derivatives, pharmaceutically acceptable salts and/or solvates thereof. Most preferably, the at least one amino acid is leucine, aspartic acid, or mixtures thereof.

The at least one amino acid may be provided in enantiomerically pure (or enantiopure) form. That is, the inhalable formulation comprises only one enantiomer of the at least one amino acid. The amino acid may be considered enantiomerically pure if is contains no more than 1% by weight of amino acid of the alternative enantiomer of the amino acid. Most preferably, the at least one amino acid contains no more than 0.1% by weight of the amino acid, most preferably no more than 0.01% by weight of the amino acid, of the alternative enantiomer of the amino acid. Preferably, the at least amino acid is provided as the L-enantiomer. Alternatively, the at least one amino acid is provided as the D-enantiomer. Alternatively, the at least one amino acid may be provided as a racemic mixture.

Preferably, the inhalable formulation according to the present invention comprises between about 0.005M and about 5M of the at least one amino acid. It will be understood that molar concentration (M) is a common parameter used in the art to measure the concentration of compounds in solution. Molar concentration used herein has its ordinary meaning to one skilled in the art, which is that the molar concentration is equal to the moles of solute divided by the litres of solution in which the solute is dissolved (i.e. mol/L). It will be understood that the concentration ranges provided herein in relation to the at least one amino acid refer to the concentration of each amino acid present in the formulation. That is, wherein the formulation comprises more than one amino acid, each amino acid is provided in a concentration of between about 0.005M and about 5M. Alternatively, the concentration ranges provided herein in relation to the at least one amino acid may refer to the concentration of all amino acids present, i.e. the total concentration of each amino acid present in the formulation. That is, wherein the formulation comprises more than one amino acid, the amino acids are provided in a total concentration of between about 0.005M and about 5M. Preferably the at least one amino acid is provided in the formulation in a concentration of between about 0.005M and about 1M, preferably the at least one amino acid is provided in a the formulation in a concentration of between about 0.05M and about 0.5M. Most preferably, the at least one amino acid is provided in the formulation in a concentration of between about 0.01M and about 0.1M. Preferably, the at least one amino acid is provided in the formulation in a concentration of at least about 0.01M. Alternatively, the at least one amino acid is provided in the formulation in a concentration of no more than about 0.5M, most preferably no more than 0.1M.

In embodiments of the invention, the formulation may further comprise an acid which is not an amino acid. For example, the formulation may comprise a mineral acid such as hydrochloric acid, nitric acid or phosphoric acid, or mixtures thereof. The formulation may comprise an organic acid, such as maleic acid. In alternative embodiments, the formulation may only comprise the amino acid as the acidic component (excluding any active ingredient with an acidic component, such as the beta2-agonist, steroid or muscarinic antagonist). For example, the formulation may not comprise a mineral acid, such as hydrochloric acid, nitric acid or phosphoric acid, or mixtures thereof, and/or an organic acid, such as maleic acid.

### Beta2-Agonist

The inhalable formulation of the present invention comprises at least one beta2-agonist. The inhalable formulation may comprise one beta2-agonist, or may comprise a combination of two or more beta2-agonists.

A beta2-agonist acts directly on beta2-receptors, causing smooth muscle relaxation and dilation of the airways. The one or more beta2-agonist may be a short-acting beta2-agonist (referred to as SABAs) or a long-acting beta2-agonist (referred to as LABAs). In some embodiments, a combination of one or more short-acting beta2-agonists and one or more long-acting beta2-agonists may be used in the inhalable formulation. According to the United Kingdom's National Institute for Health and Care Excellence (NICE), short-acting beta2-agonists have a rapid onset of action (within 15 minutes) and their effects last for up to 4 hours. Examples of short-acting beta2-agonists are salbutamol and terbutaline. According to NICE, long-acting beta2-agonists have prolonged receptor occupancy. Examples of long-acting beta2-agonists are salmeterol and formoterol. Salmeterol and formoterol are relatively lipophilic and have a duration of action of about 12 hours. Thus, embodiments of the invention may comprise at least one short acting beta2-agonist. Alternatively, embodiments of the invention may comprise at least one long-acting beta2-agonist. Alternatively, embodiments of the invention may comprise a mixture of short-acting and long-acting beta2-agonists.

The at least one beta2-agonist may comprise a benzylic hydroxyl moiety according to structural Formula I:

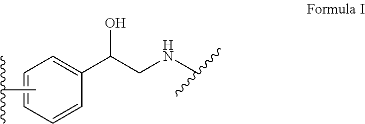

wherein

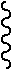

represents any functional group or chemical moiety. Thus, according to Formula I, any functional group of chemical moiety may be present in any position of the benzyl group. Additionally, any functional group or chemical moiety may be present adjacent the amine group shown in Formula I. Many beta2-agonists comprise a benzylic hydroxyl moiety according to Formula I, for example the beta2-agonists listed in Table 1. Without wishing to be bound by theory, it is believed that this benzylic hydroxyl moiety leads to chemical instability of the beta2-agonist. The inherent instability of the beta2-agonist may make the beta2-agonist particularly unstable when the beta2-agonists is in formulation, such as with a propellant. A greater instability may exist when the propellant is a HFA propellant because of the high polarity of HFA propellants. It is believed that addition of at least one amino acid to a formulation comprising a propellant and a beta2-agonist having a benzylic hydroxyl moiety according to Formula I, stabilises the benzylic hydroxyl functionality of the beta2-agonists. This is thought to be, at least in part, due to the zwitterionic nature of the amino acid which stabilises the formulation pH and the benzylic hydroxyl functionality. It may be that the amino acid also plays a role in stabilising the amine in the benzylic hydroxyl moiety according to Formula I.

Preferably, the alcohol and amine functional groups according to Formula I are both protonated, as shown in Formula I. Alternatively, the alcohol of Formula I may be substituted by group R, and/or the amine of Formula I may be substituted by group R′, as show in Formula I′. Preferably, R is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, or combinations thereof. Preferably, R′ is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, acyl, acyloxy, alkoxycarbonyl, halo, or hydroxyl or combinations thereof. Preferably, wherein R is hydrogen, R′ cannot be hydrogen. Preferably, wherein R′ is hydrogen, R cannot be hydrogen.

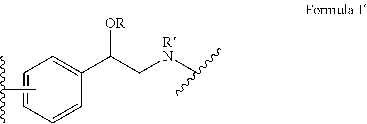

The beta2-agonist may comprise a benzylic hydroxyl moiety according to structural Formula II:

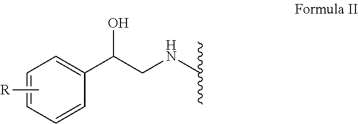

It will be understood that Formula II is a subset of Formula I. In Formula IL, the one or more functional groups or chemical moieties present on the benzyl group is represented by R. R may be located in any position on the benzyl group and the benzyl group may contain multiple R groups. Preferably, R is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, or hydroxyl or combinations thereof.

Preferably, the alcohol and amine functional groups according to Formula II are both protonated, as shown in Formula II. Alternatively, the alcohol of Formula II may be substituted by group R¹, and/or the amine of Formula II may be substituted by group R², as show in Formula II′. Preferably, R¹ is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, or combinations thereof. Preferably, R² is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, acyl, acyloxy, alkoxycarbonyl, halo, or hydroxyl or combinations thereof. Preferably, wherein R¹ is hydrogen, R² cannot be hydrogen. Preferably, wherein R² is hydrogen, R¹ cannot be hydrogen.

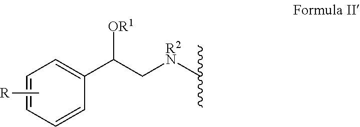

The beta2-agonist may comprise a benzylic hydroxyl moiety according to structural Formula III:

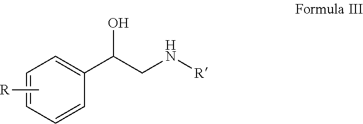

It will be understood that Formula III is a subset of Formula II. In Formula III, the one or more functional groups or chemical moieties present on the amine group is represented by R′. R may be located in any position on the benzyl group and the benzyl group may contain multiple R groups. Preferably, R is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, hydroxyl. Preferably, R′ is hydrogen, alkyl, heteroalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, hydroxyl.

Preferably, the alcohol and amine functional groups according to Formula III are both protonated, as shown in Formula III. Alternatively, the alcohol of Formula III may be substituted by group R¹, and/or the amine of Formula III may be substituted by group R², as show in Formula III′. Preferably, R¹ is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, amino, acyl, acyloxy, alkoxycarbonyl, halo, or combinations thereof. Preferably, R² is hydrogen, alkyl, heteroalkyl, haloalkyl, alkenyl, alkynyl, cycloalkyl, heterocycloalkyl, aryl, arylalkyl, heteroaryl, heteroarylalkyl, alkoxy, heterocycloalkyloxy, amido, acyl, acyloxy, alkoxycarbonyl, halo, or hydroxyl or combinations thereof. Preferably, wherein R¹ is hydrogen, R² cannot be hydrogen. Preferably, wherein R² is hydrogen, R¹ cannot be hydrogen.

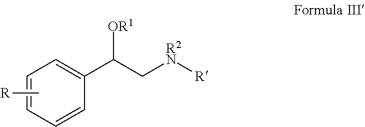

It is also envisaged that any beta2-agonists having the benzylic hydroxyl moiety according to Formulas I, I′, IL, II′, III and III′ may be suitably provided in the inhalable formulation according to the present invention.

The at least one beta2-agonist may be selected from the list of: formoterol, indacaterol, olodaterol, salmeterol, carmoterol, and/or vilanterol, and derivatives, pharmaceutically acceptable salts and/or solvates thereof. Such beta2-agonists are readily available to a person skilled in the art. The structural formula of each of these beta2-agonists is represented in Table 1. Suitable pharmaceutically salts include, but are not limited to, chloride, bromide and sulphate, phosphate, maleate, fumarate, tartrate, citrate, benzoate, mesilate, ascorbate, salicylate, acetate, succinate, lactate, glutarate or gluconate and equivalents thereof. The formulation may comprise one beta2-agonist selected from this list, or multiple beta2-agonists selected from this list. Such beta2-agonists comprise the benzylic hydroxyl moiety according to Formulas I, II and III. As such, these beta2-agonists may be classified as belonging to the phenylalkylamino class of long-acting beta2-agonists. It is envisaged that any beta2-agonist belonging to the phenylalkylamino class may be suitably provided in the inhalable formulation according to the present invention.

TABLE 1
Structural formula of beta2-agonists

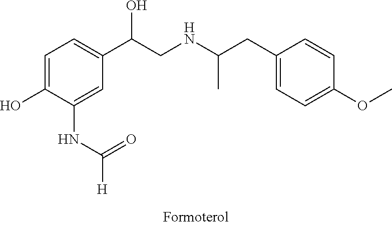 [SMILES: COc1ccc(CC(C)NCC(O)c2ccc(O)c(NC=O)c2)cc1]

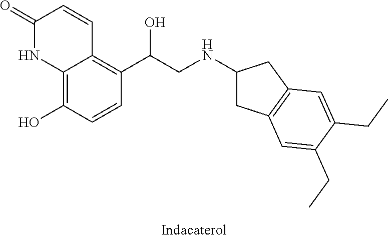 [SMILES: CCc1cc2c(cc1CC)CC(NCC(O)c1ccc(O)c3[nH]c(=O)ccc13)C2]

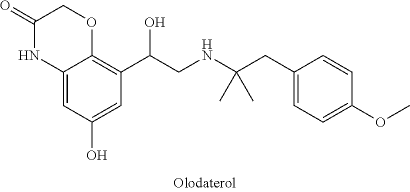 [SMILES: COc1ccc(CC(C)(C)NCC(O)c2cc(O)cc3c2OCC(=O)N3)cc1]

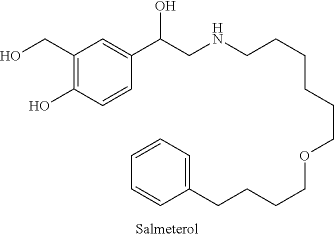 [SMILES: OCc1cc(C(O)CNCCCCCCOCCCCc2ccccc2)ccc1O]

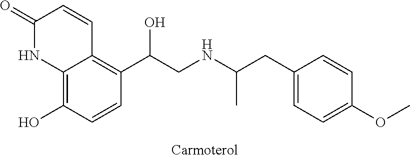 [SMILES: COc1ccc(CC(C)NCC(O)c2ccc(O)c3[nH]c(=O)ccc23)cc1]

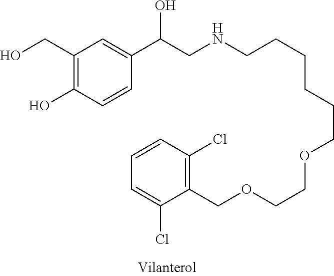 [SMILES: OCc1cc(C(O)CNCCCCCCOCCOCc2c(Cl)cccc2Cl)ccc1O]

Most preferably, the formulation comprises formoterol or a derivative, pharmaceutically acceptable salt and/or solvate thereof. Formoterol may alternatively be referred to as eformoterol. Formoterol is an example of a beta2-agonist and is effective in the treatment of asthma and lung disease such as chronic obstructive pulmonary disease (CPD) such as chronic bronchitis and emphysema. Formoterol may be provided in the inhalable formulation according to the present invention in the form of a pharmaceutically salt, such as formoterol fumarate. Formoterol fumarate may be provided in the form of formoterol fumarate dihydrate.

The formulation may comprise at least one beta2-agonist in an amount such that between about 1 μg and 50 μg of beta-2-agonist are delivered in a single actuation of a MDI loaded with the formulation, such as between 2 μg and 30 μg, for example between 4 μg and 20 μg. Preferably, the formulation comprises at least one beta2-agonist in an amount such that between about 6 μg and 12 μg of beta-2-agonist are delivered in a single actuation of a MDI loaded with the formulation. For example, the concentration of formoterol in the formulation may be such that when a MDI is loaded with the formulation, a single actuation delivers the amounts specified above, preferably between about 6 μg and about 12 μg, of formoterol. If more than one beta2-agonist is present in the formulation, each beta2 agonist is present in an amount such that that amounts specified above, such as between about 1 μg and about 50 μg, of each beta2-agonist are delivered in a single actuation of a MDI loaded with the formulation. Preferably, the amount of each beta2-agonist present in the formulation is between about 0.005% and 0.1% w/w.

### Propellant

The inhalable formulation of the present invention comprises a propellant. A propellant allows delivery of the formulation in a metered dose inhaler (MDI) system as the propellant provides the required pressure to atomize the inhalable medicinal formulation into micron-scaled droplets suitable for inhalation.

The propellant may be any pharmaceutically acceptable propellant or mixtures of two or more pharmaceutically acceptable propellants. Preferably, the propellant may comprise a fluorinated propellant. Most preferably, the propellant comprises at least one hydrofluroalkane (HFA). Hydrofluroalkane propellants are also referred to as hydrofluorocarbon propellants. The terms hydrofluoroalkane and hydrofluorocarbon are interchangeable, for example herein.

Preferably, the at least one HFA is selected from the list of HFA 134a, HFA 152a, or HFA 227 and mixtures thereof. The IUPAC name of HFA 134a is 1,1,1,2-tetrafluoroethane. The IUPAC name of HFA 152a is 1,1-difluoroethane. The IUPAC name of HFA 227 is 1,1,1,2,3,3,3-heptafluoro-n-propane. Most preferably, the propellant according to the present invention comprises HFA134a.

Beta2-agonists are known to be unstable in formulations comprising propellants, particularly over extended periods of time. This is particularly so in HFA propellant formulations owing to the high polarity of the HFA propellant. Thus, the present inhalable formulations provide at least one amino acid to stabilise the at least one beta2-agonist in formulation.

Preferably, the formulation comprises between about 80% and 99% w/w of propellant, more preferably the formulation comprises between about 90% and about 99% w/w of propellant. When more than one propellant is present in the formulation, the total amount of all propellants is between about 80% and about 99% w/w of propellant, most preferably, between about 90% and about 99% w/w of propellant.

Preferably, wherein the propellant is HFA, the formulation comprises between 80% and 99% w/w of HFA, most preferably, between about 90% and about 99% w/w of propellant.

### Steroids

Optionally, the inhalable formulation of the present invention further comprises at least one steroid. Preferably, the at least one steroid comprises one or more inhaled corticosteroids (ICS). A combination of at least one beta2-agonist, particularly a long-acting beta2-agonist, with a steroid, particularly an inhaled corticosteroid, may be particularly effective for treating a lung disease and/or disease of the respiratory tract.

Suitable corticosteroids include, but are not limited to budesonide, beclometasone, ciclesonide, fluticasone propionate, mometasone furoate. A combination of a long-acting beta2-agonist and corticosteroid provides optimal control of asthma in most patients. Particularly advantageous is a combination of formoterol, such as formoterol fumarate (for example provided in the form of formoterol fumarate dehydrate), and beclometasone dipropionate. Optionally, the formulation may further comprise a muscarinic antagonist as described herein, to provide particularly effective formulations for treatment of respiratory diseases.

Preferably, the at least one inhaled corticosteroid is selected from the list of: beclometasone, budesonide, ciclesonide, fluticasone and mometasone, and derivative, pharmaceutically acceptable salt and/or solvate and combinations thereof. Such ICSs are particularly effective for use in the treatment of asthma. Most preferably the at least one steroid is beclometasone or a derivative, pharmaceutically acceptable salt and/or solvate and combination thereof. For example, the formulation may comprise beclometasone diproprionate.

It may be that the at least one steroid is stable in formulation, thus the at least one amino acid has minimal or no effect on the stability of the at least one steroid.

The formulation may comprise at least one steroid in an amount such that between about 10 μg and about 1000 μg of the at least one steroid, such as between about 30 μg and about 800 μg, for example between about 100 μg and about 500 μg, are delivered in a single actuation of a MDI loaded with the formulation. Preferably, the formulation comprises at least one steroid in an amount such that between about 100 μg and about 200 μg of the at least one steroid are delivered in a single actuation of a MDI loaded with the formulation. For example, the concentration of beclometasone dipropionate in the formulation may be such that when a MDI is loaded with the formulation, a single actuation delivers the amounts specified above, preferably between about 100 μg and about 200 μg, of beclometasone dipropionate. If more than one steroid is present in the formulation, each steroid is present in an amount such that the amounts specified above, i.e. between about 10 μg and about 1000 μg, for example between about 100 μg and about 200 μg, of each steroid are delivered in a single actuation of a MDI loaded with the formulation. Preferably, the amount of each steroid present in the formulation is between about 0.01% and 0.5% w/w.

### Muscarinic Antagonist

Optionally, the inhalable formulation of the present invention further comprises at least one muscarinic antagonist. Muscarinic antagonist are commonly also referred to as antimuscarinic agents. The terms muscarinic antagonist and antimuscarinic agent are interchangeable, for example herein. Muscarinic agents trigger bronchodilation.

The at least on muscarinic antagonist may comprise a short acting muscarinic antagonists (e.g. ipratropium bromide); and/or a long acting muscarinic antagonists (e.g. tiotropium bromide, aclidinium bromide, glycopyrronium bromide, umeclidinium) or combinations thereof. A combination of at least one long-acting beta2-agonist, at least one corticosteroid and at least one muscarinic antagonist may be particularly advantageous for treatment of a respiratory disease.

Preferably, the at least one muscarinic antagonist is one or more long acting muscarinic antagonists selected from the list of: ipratropium bromide, oxitropium bromide, tiotropium bromide, aclidinium bromide, glycopyrronium bromide, and umeclidinium bromide, and derivatives, pharmaceutically acceptable salts and/or solvates thereof. Particularly advantageous is a combination of formoterol (for example formoterol fumarate, preferably formoterol fumarate dihydrate), beclometasone diproprionate and at least one muscarinic antagonist selected from that list.

It may be that the at least one muscarinic antagonist is stable in formulation, thus the at least one amino acid has minimal or no effect on the stability of the at least one muscarinic antagonist.

The formulation may comprise at least one muscarinic antagonist in an amount such that between about 1 μg and about 200 μg, such as between about 2 μg and about 100 μg, preferably between about 5 μg and about 50 μg of the at least one muscarinic antagonist are delivered in a single actuation of a MDI loaded with the formulation. If more than one muscarinic antagonist is present in the formulation, each muscarinic antagonist may be present in an amount such that between about 1 μg and about 200 μg, preferably between about 5 μg and about 50 μg, of each muscarinic antagonist are delivered in a single actuation of a MDI loaded with the formulation.

### Co-Solvent

Optionally, the inhalable formulation of the present invention further comprises a co-solvent or mixture of co-solvents. Preferably, the co-solvent or mixture of co-solvents is a pharmaceutically acceptable co-solvent. Most preferably, the co-solvent or mixtures of co-solvents comprises an alcohol or mixture of alcohols, for example a lower alkyl (C1-C4) alcohol, polyols, polyalkylene glycols, (poly)alkoxy derivatives or mixtures thereof. Preferably, the co-solvent is ethanol or a mixture of co-solvents comprising ethanol.

Preferably, the co-solvent is present in the formulation in an amount of between about 5% and 25% w/w, most preferably between an amount of about 8% and about 15% w/w. For example, ethanol may be present in the formulation in an amount of about 12% w/w. When more than one co-solvent is present, the formulation comprises a total amount of co-solvent within those ranges, for example the total amount of co-solvent in the formulation is between about 5% and 25% w/w.

### Disease to be Treated

The inhalable formulation of the present invention may be particularly effective in treating a disease of the lungs and/or respiratory tract. The inhalable formulation may advantageously be delivered through a pMDI.

The inhalable formulation may be particularly advantageous for use in the treatment of severe broncho-pulmonary disease. Preferably, the inhalable formulation is for use in the treatment of asthma or chronic obstructive pulmonary disease (COPD) such as bronchitis. Other respiratory disorders characterised by obstruction of the peripheral airways as a result of inflammation and presence of mucus may also be treated with the inhalable formulation according to the present invention. The formulation, use or method of the second aspect of the invention may thus be directed to the treatment or prevention, preferably the treatment, of the above mentioned disorders.

It is acknowledged that in some jurisdictions, claims to methods of treating or preventing a disorder may be regarded as patentable subject matter whereas in other jurisdictions such methods may be regarded as excluded subject matter. In this regard, the applicant reserves the right to amend the claims to comply with jurisdiction-specific subject matter requirements. For example, it is envisaged claiming a method of treating or preventing a disease of the respiratory tract and/or lungs using a formulation according to any embodiment of any aspect of the invention in jurisdictions where it is permitted to do so. For example, such method may comprise administering a dose of an inhalable formulation according to the present invention to a subject in need thereof, for example via a pMDI.

### Pressurised Metered Dose Inhalers

Inhalable formulations of the invention are particularly suited for delivery to the respiratory tract and lungs through use of a pressurized metered dose inhaler (pMDIs). pMDIs are capable of providing formulations to the lung in accurately measured doses and allow for co-deposition of multiple drugs into the lungs. It will be understood that the term “dose” means the amount of active ingredient delivered in a single actuation of the inhaler. Multiple actuations of the inhaler may be triggered sequentially or intermittently. For example, one or two activations may be used to deliver a dose e.g. a daily dose

Such inhalers comprise a pressurized canister that upon activation releases the inhalable formulation. The amount of formulation released is regulated by a metered valve capable of delivering an accurate volume of composition from the inhaler so that a controlled dose is delivered to the lungs of a patient. Thus, according to the present invention is provided a pressurized canister for use in a pressurised metered dose inhaler, the canister being pressurized with the formulation according any embodiment of any aspect of the invention. Further provided is a pressurised metered dose inhaler comprising the pressurized canister.

The canister may optionally be fitted with a suitable metering valve for regulating the dose of formulation emitted upon activation.

Embodiments of the invention are further disclosed in the following numbered clauses:

Clause 1. An inhalable formulation comprising at least one amino acid, at least one beta2-agonist comprising a benzylic hydroxyl moiety according to the structural formula:

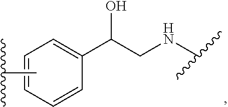

and a propellant.

Clause 2. The inhalable formulation according to clause 1, wherein the at least one amino acid is one or more amino acid selected from the list of: aspartic acid, leucine, isoleucine, alanine, and/or valine, and derivatives, pharmaceutically acceptable salts and/or solvates thereof.

Clause 3. The inhalable formulation according to clause 1 or clause 2, wherein the at least one beta2-agonist is selected from the list of: formoterol, indacaterol, olodaterol, salmeterol, carmoterol, and/or vilanterol, and derivatives, pharmaceutically acceptable salts and/or solvates thereof.

Clause 4. The inhalable formulation of clause 3, wherein the at least one beta2-agonist includes formoterol or a derivative, pharmaceutically acceptable salt and/or solvate thereof.

Clause 5. The inhalable formulation according to any preceding clause, wherein the propellant comprises at least one hydrofluroalkane (HFA).

Clause 6. The inhalable formulation according to clause 5, wherein the hydrofluroalkane is selected from the list of: HFA 134a, HFA 152a, or HFA 227, and combinations thereof.

Clause 7. The inhalable formulation according to any preceding clause further comprising at least one steroid, preferably wherein the at least one steroid is one or more inhaled corticosteroids (ICS).

Clause 8. The inhalable formulation according to clause 7, wherein the at least one steroid includes beclometasone or a derivative, pharmaceutically acceptable salt and/or solvate thereof.

Clause 9. The inhalable formulation according to any preceding clause, further comprising at least one muscarinic antagonist.

Clause 10. The inhalable formulation according to clause 9, wherein the at least one muscarinic antagonist is one or more muscarinic antagonist selected from the list of: ipratropium bromide, oxitropium bromide, tiotropium bromide, aclidinium bromide, glycopyrronium bromide, and umeclidinium bromide, and derivatives, pharmaceutically acceptable salts and/or solvates thereof.

Clause 11. The inhalable formulation according to any preceding clause, further comprising a co-solvent or mixture of co-solvents, preferably wherein the co-solvent or mixture of co-solvents is a pharmaceutically acceptable alcohol or mixture of alcohols.

Clause 12. The inhalable formulation according to clause 12, wherein the co-solvent is ethanol.

Clause 13. The inhalable formulation according to any one of clauses 1 to 12 for use in the prevention or treatment of a disease of the lungs and/or respiratory tract.

Clause 14. The inhalable formulation according to clause 13, for use in the treatment or prevention of asthma or chronic obstructive pulmonary disease (COPD).

Clause 15. A pressurized canister for use in a pressurised metered dose inhaler, the canister being pressurized with the formulation according to any one of clauses 1 to 14.

Clause 16. A pressurised metered dose inhaler comprising the pressurized canister according to clause 15.

### Examples

Ten inhalable formulations containing HFA134a, ethanol, formoterol fumarate dihydrate (FFD), beclometasone diproprionate (BDP) and an acid were prepared. Each formulation comprised 0.17% w/w of BDP, 0.0105% w/w of FFD, 12% w/w of ethanol, and 87.79% w/w of HFA. The proton donating species was varied between the ten formulations, such that each of a formulation comprising hydrochloric acid (0.1M), sulphuric acid (1.0M), citric acid (0.1M), maleic acid (1.0M), ascorbic acid (0.1M), leucine (1.0M), aspartic acid (0.1M and 0.01M) and citrate buffer (0.05M) was prepared (Formulations 1 to 10 as shown in Table 1). All other components of the formulation were kept constant in the ten formulations.

Formulations were prepared at a temperature of between 2 and 8° C. The formulations were then stored at a temperature of 40° C. and a relative humidity of 75%. These conditions are elevated over typical conditions used for storage of inhalable formulations, for example in the home. A greater stability (less degradation of active ingredient over a specified period of time) may be expected at lower temperature and lower relative humidity storage conditions, for example, at a temperature of between 2° and 25° C. and a relative humidity of less than 70%. The stability of each formulation was measured over time. In order to determine stability of the FFD in formulation, the average assay of FFD was measured using HPLC according to standard methods known to one skilled in the art, immediately after the formulation was prepared, after 2 weeks of storage of the formulation, and after 4 weeks of storage of the formulation. The stability of BDP in the formulation was also determined at the same time points. The average assay of FFD and BDP at each time point is shown in Table 2.

As can be seen in Table 2, significant degradation of FFD occurred in formulations 2, 3, 4 and 6 over the 4 week period. Formulation 2, comprising sulphuric acid (1.0M) contained significant impurities of FFD immediately after formulation. It can be inferred that sulfuric acid, citric acid, ascorbic acid and citrate buffer are poor stabilisers of FFD in the inhalable formulation. In particular, formulations containing sulphuric, citric and ascorbic acid resulted in significant degradation of formoterol as indicated by the large impurity profile after 4 weeks of storage of formulations 2, 3, 4 and 6.

Formulation 1 (containing HCl 0.1M), formulation 5 (containing maleic acid 1.0M), and formulations 7, 8 and 9 comprising leucine or aspartic acid, stabilised the FFD component of the formulation over the 4 week period. After 4 weeks of storage, there was a slight increase of the FFD impurities for the formulation containing HCl (formulation 1), but the impurities remained below 1%. Amino acids such as leucine and aspartic acid are biocompatible and therefore may be preferable for use in inhalable formulations over hydrochloric acid and maleic acid which are either detrimental to health, particularly lung health, or their impact on health is unknown.

Formulations 7, 8 and 9 containing leucine or aspartic acid showed low levels of formoterol impurities over 4 weeks which demonstrates that amino acids are able to stabilise formoterol in MDI formulations. Without wishing to be bound by theory, it is believed that the zwitterionic properties of amino acids control the pH and proton acceptor/donor profile in the formulation.

BDP was not significantly degraded in any formulation suggesting that the acid component in the formulation does not play a role, or plays a minimal role, in stabilising BDP.

TABLE 2
Assay and impurities profile of beclomethasone and formoterol MDI formulations containing different acids.

| | | | | | | | Average | | Average |
|---|---|---|---|---|---|---|---|---|---|
| | | | Time- | | | Average | total | Average | total |
| | | | point | | Average Water | Assay FFD | impurities | Assay BDP | impurities |
| Formulation | Acid | pH | (weeks) | Condition | content (ppm) | (Area %) | FFD (%) | (Area %) | BDP (%) |
| 1 | HCl 0.1M | 5.66 ± 0.16 | 0 | 2-8° C. | 2395.63 ± 94.45 | 99.80 ± 0.28 | 0.20 ± 0.28 | 99.90 ± 0.04 | 0.10 ± 0.04 |
| | | — | 2 | 40° C./75% | — | 96.34 ± 4.03 | 0.81 ± 0.01 | 99.59 ± 0.07 | 0.41 ± 0.07 |
| | | — | 4 | RH | — | 99.11 ± 0.05 | 0.89 ± 0.05 | 99.90 ± 0.01 | 0.10 ± 0.01 |
| 2 | Sulfuric | 3.14 ± 0.21 | 0 | 2-8° C. | 458.9 ± 23.81 | 85.21 ± 0.41 | 14.79 ± 0.41 | 99.66 ± 0.10 | 0.34 ± 0.10 |
| | acid | — | 2 | 40° C./75% | — | 43.62 ± 0.00 | 56.38 ± 0.00 | 99.97 ± 0.00 | 0.03 ± 0.00 |
| | 1.0M | — | 4 | RH | — | 36.46 ± 4.90 | 63.54 ± 4.90 | 99.92 ± 0.07 | 0.08 ± 0.07 |
| 3 | Citric | 6.39 ± 0.37 | 0 | 2-8° C. | 339.97 ± 3.40 | 99.86 ± 0.10 | 0.14 ± 0.10 | 99.96 ± 0.02 | 0.04 ± 0.02 |
| | acid | — | 2 | 40° C./75% | — | 95.62 ± 0.56 | 4.38 ± 0.56 | 99.82 ± 0.14 | 0.18 ± 0.14 |
| | 1.0M | — | 4 | RH | — | 86.63 ± 7.16 | 13.37 ± 7.16 | 99.81 ± 0.00 | 0.19 ± 0.00 |
| 4 | Citric | 6.51 ± 0.10 | 0 | 2-8° C. | 1272.27 ± 35.73 | 99.36 ± 0.02 | 0.64 ± 0.02 | 99.96 ± 0.02 | 0.04 ± 0.02 |
| | acid | — | 2 | 40° C./75% | — | 95.11 ± 0.20 | 4.89 ± 0.20 | 99.85 ± 0.00 | 0.15 ± 0.00 |
| | 0.1M | — | 4 | RH | — | 93.64 ± 0.10 | 6.36 ± 0.10 | 99.94 ± 0.01 | 0.06 ± 0.01 |
| 5 | Maleic | 4.69 ± 0.01 | 0 | 2-8° C. | 590.27 ± 14.99 | 98.54 ± 1.56 | 1.46 ± 1.56 | 99.76 ± 0.23 | 0.24 ± 0.23 |
| | acid | — | 2 | 40° C./75% | — | 98.37 ± 0.30 | 1.63 ± 0.30 | 99.97 ± 0.00 | 0.03 ± 0.00 |
| | 1.0M | — | 4 | RH | — | 98.53 ± 0.69 | 1.47 ± 0.69 | 99.99 ± 0.01 | 0.01 ± 0.01 |
| 6 | Ascorbic | 6.27 ± 0.03 | 0 | 2-8° C. | 5362.17 ± 195.29 | 98.47 ± 2.66 | 0.00 ± 0.00 | 99.76 ± 0.14 | 0.24 ± 0.14 |
| | acid | — | 2 | 40° C./75% | — | 93.44 ± 0.23 | 6.56 ± 0.23 | 99.77 ± 0.02 | 0.23 ± 0.02 |
| | 0.1M | — | 4 | RH | — | 98.27 ± 2.29 | 1.73 ± 2.29 | 99.69 ± 0.29 | 0.31 ± 0.29 |
| 7 | Leucine | 6.85 ± 0.52 | 0 | 2-8° C. | 1288.6 ± 21.62 | 98.85 ± 0.05 | 1.15 ± 0.05 | 99.80 ± 0.02 | 0.20 ± 0.02 |
| | 1.0M | — | 2 | 40° C./75% | — | 99.01 ± 0.10 | 0.99 ± 0.10 | 99.94 ± 0.01 | 0.06 ± 0.01 |
| | | — | 4 | RH | — | 98.43 ± 0.67 | 1.57 ± 0.67 | 99.82 ± 0.04 | 0.18 ± 0.04 |
| 8 | Aspartic | 6.99 ± 0.37 | 0 | 2-8° C. | 2351.7 ± 121.07 | 100.00 ± 0.00 | 0.00 ± 0.00 | 100.00 ± 0.00 | 0.00 ± 0.00 |
| | acid | — | 2 | 40° C./75% | — | 99.26 ± 0.07 | 0.74 ± 0.07 | 99.96 ± 0.00 | 0.04 ± 0.00 |
| | 0.1M | — | 4 | RH | — | 99.48 ± 0.01 | 0.52 ± 0.01 | 99.97 ± 0.01 | 0.03 ± 0.01 |
| 9 | Aspartic | 7.13 ± 0.05 | 0 | 2-8° C. | 452.57 ± 20.34 | 99.92 ± 0.11 | 0.08 ± 0.11 | 99.93 ± 0.05 | 0.07 ± 0.05 |
| | acid | — | 2 | 40° C./75% | — | 99.31 ± 0.00 | 0.69 ± 0.00 | 99.84 ± 0.15 | 0.16 ± 0.15 |
| | 0.01M | — | 4 | RH | — | 99.56 ± 0.06 | 0.44 ± 0.06 | 99.94 ± 0.00 | 0.06 ± 0.00 |
| 10 | Citrate | 4.56 ± 0.59 | 0 | 2-8° C. | 2347.8 ± 46.44 | 99.81 ± 0.17 | 0.19 ± 0.17 | 99.86 ± 0.09 | 0.14 ± 0.09 |
| | buffer | — | 2 | 40° C./75% | — | 98.35 ± 0.16 | 1.65 ± 0.16 | 100.00 ± 0.00 | 0.00 ± 0.00 |
| | 0.05M | — | 4 | RH | — | 97.48 ± 0.04 | 2.52 ± 0.04 | 99.97 ± 0.00 | 0.03 ± 0.00 |

Wherein the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. An inhalable formulation comprising at least one amino acid, at least one beta2-agonist comprising a benzylic hydroxyl moiety according to the structural formula: 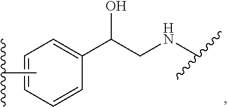
and a propellant;
wherein the inhalable formulation is a solution formulation in which the at least one beta2-agonist is dissolved in solution.

2. The inhalable formulation according to claim 1, wherein the at least one amino acid is one or more amino acid selected from the list of: aspartic acid, leucine, isoleucine, alanine, and/or valine, and derivatives, pharmaceutically acceptable salts and/or solvates thereof.

3. The inhalable formulation according to claim 1, wherein the at least one beta2-agonist is selected from the list of: formoterol, indacaterol, olodaterol, salmeterol, carmoterol, and/or vilanterol, and derivatives, pharmaceutically acceptable salts and/or solvates thereof.

4. The inhalable formulation of claim 3, wherein the at least one beta2-agonist includes formoterol or a derivative, pharmaceutically acceptable salt and/or solvate thereof.

5. The inhalable formulation according to claim 1, wherein the propellant comprises at least one hydrofluroalkane (HFA).

6. The inhalable formulation according to claim 5, wherein the hydrofluroalkane is selected from the list of: HFA 134a, HFA 152a, or HFA 227, and combinations thereof.

7. The inhalable formulation according to claim 1, further comprising at least one steroid.

8. The inhalable formulation according to claim 7, wherein the at least one steroid includes beclometasone or a derivative, pharmaceutically acceptable salt and/or solvate thereof.

9. The inhalable formulation according to claim 1, further comprising at least one muscarinic antagonist.

10. The inhalable formulation according to claim 9, wherein the at least one muscarinic antagonist is one or more muscarinic antagonist selected from the list of: ipratropium bromide, oxitropium bromide, tiotropium bromide, aclidinium bromide, glycopyrronium bromide, and umeclidinium bromide, and derivatives, pharmaceutically acceptable salts and/or solvates thereof.

11. The inhalable formulation according to claim 1, further comprising a co-solvent or mixture of co-solvents.

12. The inhalable formulation according to claim 11, wherein the co-solvent is ethanol.

13. A method of prevention or treatment of a disease of the lungs and/or respiratory tracts, said method comprising administering the inhalable formulation according to claim 1 to a person in need thereof.

14. The method according to claim 13, wherein the disease is asthma or chronic obstructive pulmonary disease (COPD).

15. A pressurized canister for use in a pressurised metered dose inhaler, the canister being pressurized with the formulation according to claim 1.

16. A pressurised metered dose inhaler comprising the pressurized canister according to claim 15.

17. The inhalable formulation according to claim 7, wherein the at least one steroid is one or more inhaled corticosteroids (ICS).

18. The inhalable formulation according to claim 11, wherein the co-solvent or mixture of co-solvents is a pharmaceutically acceptable alcohol or mixture of alcohols.
